(19) United States
(12) Patent Application Publication
(10) Pub. No.: US 2009/0156690 A1
(43) Pub. Date: Jun. 18, 2009

(54) Deodorizing Exfoliator

(76) Inventors: S. Bruce Hardwick, JR., Colusa, CA (US); Jennifer L. Hardwick, (US)

Correspondence Address: Ken Campbell, PO Box 551, Fair Oaks, CA, 95628, US

(21) Appl. No.: 11/955,059

(22) Filed: Dec. 12, 2007

(51) Int. Cl. A61K 31/01

(52) U.S. Cl. 514/762

(57) ABSTRACT

Disclosed is a method of treating body odor by scrubbing onto the skin a mixture of salt, one or more humectants, and one or more essential oils, with the salt being a gritty solid component in suspension within the other components so that the mixture is a viscously heterogeneous composition, which feels gritty to the touch and which exfoliates and moisturizes the skin when the mixture is scrubbed onto the skin, where the scrubbing allows the humectant(s) to penetrate into the skin. After scrubbing, the mixture is rinsed off of the skin (particularly the salt) with water, leaving a portion of mixture (minus the salt) remaining on the skin to terminate any residual fungi or bacteria, and to continue inhibiting the growth of new fungi and bacteria, while giving the skin a fragrance.

### BACKGROUND OF THE INVENTION

There has never been a skin product that does all of the following things:
It provides a composite mixture of humectants, an abrasive, and bacterial/fungal inhibitors.
It contains natural salt crystals as an exfoliate, suspended in a viscously heterogeneous matrix.
It contains organic humectants to act as suspending agents, which remain on the body to moisturize the skin.
It leaves a coating of fragrant essential oils on the surface of the skin.
It acts as a concentrated antimicrobial agent to inhibit the growth of fungi and bacteria, and to prevent the development of odor.
It is completely organic.
It has no preservatives.
It is non-toxic.
It is formulated to accomplish its intended purposes with a simple application.

### SUMMARY

Through the deliberate physical processes resulting from its use, this invention effectively eliminates body odor. It is not a deodorant. It is not an antiperspirant. It is a deodorizing exfoliator. With unsurpassed simplicity, this inventive product is applied as a scrub (Step 1), and rinsed (Step 2).

The first step in this procedure mechanically separates the dead cells and bacterial growth from the living skin. The second step removes the waste and the suspended crystals, while leaving the matrix on the skin. This matrix then naturally diverges into its useful components. The humectants get steadily absorbed into the skin, acting to moisturize it. Meanwhile, the natural antimicrobial oils remain on the surface of the skin in rising concentrations, to annihilate any remaining fungi or bacteria, and to inhibit their growth until the next application.

Because no new growth of fungi or bacteria takes place, body odor is prevented completely.

### DESCRIPTION

This organic mixture contains only six necessary ingredients: salt, humectants (glycerin and propylene glycol), Rosemary oil, Tea Tree oil, and Peppermint oil.

The crystals are composed of sea salt. They range in size from about 1-3 mm. It was found that, when they are initially immersed in the Tea Tree oil, it helps to protect the salt crystals, while preventing them from being dissolved in the mixture, during storage and use.

The humectants serve to suspend the salt crystals in the mixture, so that it can be used as an abrasive exfoliator. The humectants also get absorbed into the skin, and remain there after rinsing, to act as a moisturizer.

The Tea Tree oil and Peppermint oil are both natural antifungal and antiseptic agents (and essential oils). They appear to act through a mechanism that is distinct from that of antibiotics. Consequently, these agents are not susceptible to cross-resistance by the bacteria found under the arms. Nevertheless, once the humectants have been absorbed, the concentrations of these oils on the skin surface are far greater than the recommended minimum concentrations for antimicrobial dilutions (4%). While the Tea Tree oil has a clean medicine-like aroma, the Peppermint oil and the Rosemary oil compliment it nicely with a blend of pleasant smell.

If you want some of this product, you'll have to wait.

1. A method of making a deodorizing exfoliator comprising:
a first step of combining salt with oil; and a second step of adding one or more humectants, and one or more essential oils to form a mixture, the two steps performed so that the salt remains a solid component in suspension within the other components and so that the mixture is a viscously heterogeneous composition, which feels gritty to the touch and which exfoliates and moisturizes an area of skin when the mixture is scrubbed onto the area of skin; wherein the humectant(s) will absorb into the area of skin, and wherein, after the mixture is at least partly rinsed off of the skin (particularly the salt) with water, a portion of the mixture (minus the salt) remains on the skin to terminate any residual fungi or bacteria, and to continue inhibiting the growth of new fungi and bacteria, while giving the skin a fragrance.

2. A method of treating body odor comprising:
scrubbing onto the skin a mixture of salt, one or more humectants, and one or more essential oils, the salt being a gritty solid component in suspension within the other components so that the mixture is a viscously heterogeneous composition, which feels gritty to the touch and which exfoliates and moisturizes the skin when the mixture is scrubbed onto the skin, wherein the scrubbing allows the humectant(s) to penetrate into the skin; and after the step of scrubbing, at least partly rinsing the mixture off of the skin (particularly the salt) with water, leaving a portion of mixture (minus the salt) remaining on the skin to terminate any residual fungi or bacteria, and to continue inhibiting the growth of new fungi and bacteria, while giving the skin a fragrance.

3. A deodorizing exfoliator comprising:
a mixture of salt, one or more humectants, and one or more essential oils, the salt being a gritty solid component in suspension within the other components so that the mixture is a viscously heterogeneous composition, which feels gritty to the touch and which exfoliates and moisturizes the skin when the mixture is scrubbed onto the skin; wherein the scrubbing allows the humectant(s) to penetrate into the skin, and wherein, after said deodorizing exfoliator is at least partly rinsed off of the skin (particularly the salt) with water, a layer of the composition (minus the salt) remains on the skin to terminate any residual fungi or bacteria, and to continue inhibiting the growth of new fungi and bacteria, while giving the skin a fragrance.
